(19) United States
(12) Patent Application Publication
(10) Pub. No.: US 2016/0143926 A1
(43) Pub. Date: May 26, 2016

(54) COMPOSITIONS AND METHODS FOR THE PREPARATION OF KIDNEY PROTECTIVE AGENTS COMPRISING AMIFOSTINE AND AMINO ACIDS

(71) Applicant: AMINOMEDIX INC., Houston, TX (US)

(72) Inventors: Ebrahim Delpassand, Houston, TX (US); Izabela Tworowska, Houston, TX (US); Sanjay Thamake, Houston, TX (US); David Ranganathan, Houston, TX (US)

(21) Appl. No.: 14/899,082

(22) PCT Filed: Jun. 16, 2014
(86) PCT No.: PCT/US14/42535

§ 371 Date: Dec. 16, 2015

Related U.S. Application Data
(60) Provisional application No. 61/836,561, filed on Jun. 18, 2013.

(51) Int. Cl. A61K 31/661; A61K 31/714; A61K 31/4172; A61K 31/198; A61K 31/195

(52) U.S. Cl. CPC A61K 31/661; A61K 31/198; A61K 31/195; A61K 31/4172; A61K 31/714

(57) ABSTRACT

This invention relates to composition and method of preparation of AminoMedix™ comprising of Amifostine, at least one amino acid (Arginine, Lysine, Histidine) with or without other pharmaceutically active compounds. The AminoMedix™ composition can be applied for kidney protection during therapy using radiolabeled and non-radiolabeled compounds, contrast agents, chemotherapeutics, antibiotics and drugs showing nephrotoxic effect.

### TECHNICAL FIELDS

The present invention relates to compositions that comprise of Amifostine in combination with at least one amino acid, with or without one or more pharmaceutically active compounds. Compositions of the invention may be referred to as AminoMedix™ and are used to reduce nephrotoxic effects of radiolabeled and non-radiolabeled therapeutic and diagnostic compounds. Another aspect of invention relates to methods for the preparation of AminoMedix™ compositions comprising Amifostine and at least one amino acid, with or without one or more pharmaceutically active compounds. The compositions may be prepared with pharmaceutically accepted salts for intravenous injection.

### TECHNICAL BACKGROUND

Kidneys are dose-limiting organs in peptide receptor radionuclide therapies (PRRT) due to their tubular reabsorption and high retention. Nephrotoxicity is a side-effect of therapies using radiolabeled peptides, proteins, antibodies and non-radiolabeled compounds, chemotherapeutics, aminoglycoside antibiotics, and contrast agents. (Pool E, 2010; Melis M, 2005).

Aminoglycoside antibiotics and radiolabeled somatostatin analogs accumulate in the kidney via megalin and cubulin receptor-mediated endocytosis. Co-administration of megalin-targeting ligands, such as positively charged amino acids, can decrease the accumulation of these nephrotoxic compounds in renal proximal tubular cells. (Nagai, 2010; Moestrup S K, 1996; Hammond P J, 1993; Baron R, 2004; Melis M., 2012.; Vegt E. 2010).

Amifostine (also known as WR-2721) has been shown to act as kidney radioprotective agent in patients undergoing external beam radiation therapy. Amifostine is a pro-drug that can be enzymatically dephosphorylated by alkaline phosphatase to form an active metabolite, WR-1065. This process proceeds with higher rates in normal cells than in cancer cells due to higher expression of alkaline phosphatase in the healthy tissues. (Copp R R, 2013; Andreasssen C N, 2003; Santini V, 2001; Culy C R, 2001).

For example, European patent application No. EP1368038 (based on WO 2002062350 A1) describes the administration of Amifostine and/or related phosphorothioate derivatives as cytoprotective compounds during conventional external beam radiation therapy.

Amifostine administrated by i.v. injection, as a single dose of 200 mg/m², has been approved as a radioprotective agent during radiotherapy of head and neck cancers (Lindegaard, 2003). Amifostine is the only drug to have a proven radioprotective effect in patient with acute radiation-induced esophagitis (Giraud P., 2012).

Positively charged amino acids have been shown to decrease renal accumulation of radiolabeled somatostatin analogs and protect the kidneys against radiation. In rats, intravenous co-injection of L-Lysine (400 mg/kg) and ¹¹¹In-DTPA-octretide has been shown to produce 40% kidney protection (DeJong, 1996).

Intravenous injection of D-Lysine and L-Lysine has been shown to result in more than 50% kidney protection against ¹¹¹In-DTPA-octeotide renal accumulation (Betrand, 1997). Contrary to L-Lysine, D-Lysine had no effect on the retention of this tracer in Somatostatin Receptor (SSTR)-positive organs, such as pancreas and adrenal glands. Thus, D-lysine might be preferred for the use in PRRT of SSTR-positive neuroendocrine tumors.

Co-infusion of 140 g Lysine and 29 g Arginine (Aminosyn II 7%, Abbott Lab.) has been applied in Europe during PRRT therapy of NET patients using ¹⁷⁷Lu and Y⁹⁰-labelled somatostatin analogs. Combination of Lys/Arg decreased the renal uptake of these radiopeptides and reduced kidney radiation exposure by 35% (Breeman, 1996; Valkema, 2005). Amino acid solutions (2 L) have been administrated by i.v. infusion that preceded over 4 h in patients treated with Y⁹⁰-OctreoTher (Bushnell, 2004) and led to protection of kidneys by 32%.

Aminsteril N-hepa 8% (10.32 g of Lysine and 16.08 g of Arginine) decreases renal accumulation of ¹¹¹In-DTPA-octretreotide in patients by 21%±14%. In the same study, administration of 25 g of lysine protected kidney by 17%±9%, while infusion of 75 g of lysine have shown higher effects in kidney protection, 44%±11% (Rolleman 2002).

Combination of 25 g of Lysine and 25 g of Arginine during therapy using ¹¹¹In-pentetreotide led to reduction of renal concentration of this radiopeptide, resulting in kidney protection of up to 33±23% (Rolleman, 2002).

U.S. patent application publication No. 2009/0318330 A1 (based on PCT/EP00/06917) describes the radioprotective effects of Lysine (poly-lysine) in combination with Gelofusine. The composition of Gelufosine (20 mg) and Lysine (100 mg) provides kidney protection of 65±11% when administrated with ¹⁷⁷Lu-DOTA°-Tyr³]-octreotide in male Lewis rats. A combination of Gelofusine and Lysine has significantly higher radioprotective effects on kidneys than the effects of Gelofusine (20 mg) or Lysine (100 mg) used alone.

Administration of Lysine (25 g), Arginine (25 g) and Gelofusine in a patient treated with a high dose of ¹⁷⁷Lu-DOTA°-Tyr³]-octreotide led to kidney protection by additional 25%, as compared to a composition of Lys and Arg used without Gelofusine (W02007137871A1).

While these prior art approaches provide effective compositions and methods to reduce kidney damages in PRRT, more effective compositions and methods are still needed.

### SUMMARY OF THE INVENTION

One aspect of the invention relates to new compositions comprising Amifostine and at least one amino acid or its oligomer. A composition of the invention may further comprise one or more pharmaceutically active compounds. The pharmaceutically active compounds may include, but are not limited to, Vitamin B12, Carnitine, L-Histidine, D-Histidine, Probenecid, Albumin and product of its proteolysis, Globulin and product of its proteolysis, Peptide fragments of cytochrome c, Peptide fragments of actin-regulating proteins. These compositions may be used as kidney radioprotective agents during peptide receptor radionuclide therapies (PRRT).

In accordance with some embodiments of the invention, the composition comprises Amifostine in an amount of about 2 mg to about of 180 mg per kilogram of body weight and the at least one amino acid or its oligomer in an amount of about 150 mg to about 1,000 mg per kilogram of body weight.

Another aspect of the invention relates to methods using a composition of the invention comprising Amifostine and at least one amino acid, with or without one or more pharmaceutically active compounds, in imaging and/or therapy with one or more radiolabeled and/or non-radiolabeled agents. A radiolabeled agent comprises a radionuclide selected from 177Lu, 111In, 90Y, 117mSn, 45Ti, 59Fe, 60Cu, 61Cu, 62Cu, 64Cu, 67Ga, 68Ga, 89Sr, 99mTc, 149Pm, 153Gd, 153Sm, 186Re, 188Re, 211At, 212Bi, 225Ac, 125I, 123I, 32P, or 223Ra. The radiolabeled agent may be a peptide, a protein, an antibody, a carbohydrate, a glycopeptide, a urea-derivative, a nucleotide, a nucleoside, a heterocyclic compound, a plant extract, a nanoparticle, a polymer, a nanomaterial, or a composition comprising a plant-derived compound.

In accordance with some embodiments of the invention, the therapy is peptide receptor targeted therapy (PRRT) using a radiolabeled agonist or antagonist. The agonist or antagonist may target a somatostatin-receptor, wherein the agonist or antagonist is a DOTA-, DTPA-, or NOTA-based derivative. The DOTA-, DTPA-, or NOTA-based derivative may be selected from DOTATATE, DOTATOC, DOTANOC, DOTA-BASS, or DOTA-BIM.

Other aspects of the invention will become apparent with reference to the drawings and detailed description.

### BRIEF DESCRIPTION OF DRAWINGS

FIG. 1 shows a schematic illustrating a protocol for the administration of a composition of Amifostine and at least one amino acid in mice. The first i.v. injection of the composition may be scheduled 30 min prior to 68Ga-DOTATATE administration, while the next two injections may be performed at 15 min and 60 min, respectively, after the radiopeptide administration.

FIG. 2. shows results illustrating kidney protective properties of Lysine-Arginine (12.4 mg/ml and 12.5 mg/ml, respectively) in treatment with commercially available Clinisol® and Gelofusine co-administrated with ⁶⁸Ga-DOTATATE (40 uCi) in mice.

FIG. 3 shows results of ⁶⁸Ga-DOTATATE uptake in the kidneys and kidney protective properties of Lysine-Arginine (12.5 mg/ml and 12.5 mg/ml, respectively) and commercially available Clinisol® and Gelofusine. Administration of Lysine-Arginine composition (12.5 mg/ml and 12.5 mg/ml, respectively) protects kidneys by 36.45%. Clinisol® and Gelofusine decreases accumulation of the radiolabeled peptide and protects kidney by 32.12% and 57.7%, respectively.

FIG. 4 shows results illustrating kidney protective properties of Amifostine with Amino acids (Lysine and Arginine) that were co-administrated with ⁶⁸Ga-DOTATATE.

FIG. 5 shows results of ⁶⁸Ga-DOTATATE retention in the kidneys and kidney protective properties of Amifostine with Amino acids (Lysine and Arginine). Kidney protection was 57.83% in the presence of a Lysine-Arginine composition (25 mg/ml and 25 mg/ml) and 67.62% in the presence of Lysine-Arginine (25 mg/ml and 25 mg/ml) and Amifostine (0.63 mg/ml).

FIG. 6 shows results illustrating kidney radioprotective properties of Amifostine together with at least one Amino acid (Lysine and/or Arginine), with or without other pharmaceutically active compounds, co-administrated with ⁶⁸Ga-DOTATATE.

FIG. 7 shows results of ⁶⁸Ga-DOTATATE retention in the kidneys and radioprotective properties of Amifostine together with at least one Amino acid (Lysine and/or Arginine), with or without other pharmaceutically active compounds. Co-injection of Amifostine (0.63 mg/ml), Lysine (25 mg/ml), and L-Histidine (25 mg/ml) led to radioprotection of kidneys by 56.3%.

FIG. 8 shows results illustrating % kidney protection in the presence of Amifostine together with at least one amino acid and Vitamin B12, co-administered with ⁶⁸Ga-DOTATATE

FIG. 9 shows results of ⁶⁸Ga-DOTATATE retention in the kidneys and kidney protection properties of Amifostine together with at least one amino acid and VitaminB12. Administration of Amifostine (0.63 mg/ml), Lysine (25 mg/ml), L-Arginine (25 mg/ml), and Vitamin B12 led to protection of kidney by 42.64%, while Amifostine alone protected kidney only by 12.24%.

FIG. 10 shows results illustrating kidney radioprotective properties of Amifostine together with at least one amino acid, when co-administrated with ⁶⁸Ga-DOTATATE.

FIG. 11 shows results of ⁶⁸Ga-DOTATATE retention in the kidneys and kidney radioprotective properties of Amifostine together with at least one amino acid.

### DETAILED DESCRIPTION

Embodiments of the invention relate to compositions and methods for reducing kidney radiation damages in PRRT. These compositions may be referred to as AminoMedix™ and comprise Amifostine and at least one amino acid and/or its oligomer. In addition, these compositions may further comprise one or more pharmaceutically acceptable excipient, carrier, salt, diluent or a combination thereof. A composition of the invention may be prepared as a dry kit preparation for use in pharmacy or a cGMP facility

As discussed above, Amifostine has been shown to have cytoprotective effects during conventional external beam radiation therapy. However, Amifostine has not been shown to be effective in protecting tissues or organs against damages caused by therapeutic or diagnostic agents (i.e., non-external radiation) administered to a patient.

The amino acids in AminoMedix™ compositions may be natural or unnatural amino acids and may be selected from positive amino acids, such as histidine, lysine, arginine, ornithine, or a combination thereof. These amino acids may include L-amino acids, D-amino acids, or a mixture of the L- and D-amino acids. The oligomer in AminoMedix™ compositions may comprise these positively charged amino acids. The oligomers may be homo oligomers (i.e., comprising one type of amino acid, such as poly-histidine poly-lysine, or poly-arginine). Alternatively, the oligomers may be hetero oligomers (i.e., comprising two or more different types of amino acids), which may comprise all positively charged amino acids (e.g., comprising a mixture of histidine, lysine, and arginine) or may comprise a mixture of positively charged amino acids and non-positively charged amino acids.

The AminoMedix™ compositions may be used as kidney protective agents, particularly in imaging and/or therapeutic treatments using a radiolabeled compound such as a radiolabeled peptide and/or a non-radiolabeled compound. These compositions may be used with or without one or more other active compounds (e.g., other protective compounds).

The AminoMedix™ compositions may comprise a pharmaceutically active compound, such as Vitamin B12, Carnitine, L-Histidine, D-Histidine, Probenecid, Albumin and product of its proteolysis, Globulin and product of its proteolysis, Peptide fragments of cytochrome c, Peptide fragments of actin-regulating proteins. A pharmaceutically active compound as used herein refers to a compound that can further increase kidney radioprotective effect of Amifostine and at least one amino acid. The AminoMedix™ compositions may be used for kidney protection during imaging and/or therapy using compounds labeled with radioisotope. The following are non-limiting examples of different radionuclides used for this purpose, such as 177Lu, 111In, 90Y, 117mSn, 45Ti, 59Fe, 60Cu, 61Cu, 62Cu, 64Cu, 67Ga, 68Ga, 89Sr, 99mTc, 149Pm, 153Gd, 153Sm, 186Re, 188Re, 211At, 212Bi, 225Ac, 125I, 123I, 32P, 223Ra.

In accordance with embodiments of the invention, the therapy may use an antibiotic (such as, but not limited to, aminoglycosides or amphotericin B) and other non-radiolabeled nephrotoxic drugs. The therapy may be chemotherapy using a chemotherapy agent, such as but not limited to cisplatin and its derivatives.

In accordance with embodiments of the invention, the imaging may involve using a radiocontrast agent or a gadolinium-based contrast agent. The imaging may also involve using multimodality compounds, such as but not limited to radiolabeled/optical probes.

The AminoMedix™ compositions may be used for kidney protection during imaging and/or therapy using compounds such as but not-limited to peptides, proteins, antibodies, carbohydrates, glycopeptides, urea-derivatives, nucleotides, nucleosides, heterocyclic compounds, plant extracts, nanoparticles, polymers and nanomaterials, compositions of the plant-derived compounds.

The AminoMedix™ compositions may be used for kidney protection during peptide receptor radionuclide therapy (PRRT) using radiolabeled agonists and antagonists. The following are non-limiting examples of different agonists and antagonists targeting somatostatin-receptors including their DOTA- and DTPA-, NOTA-based derivatives such as, DOTATATE, DOTATOC, DOTANOC, DOTA-BASS, DOTA-BIM.

The AminoMedix™ compositions may be used for kidney protection during therapy using antibiotics (such as but not limited to aminoglycosides, amphotericin B) and other non-radiolabeled nephrotoxic drugs. The AminoMedix™ compositions may be used for kidney protection during chemotherapy using such as but not limited to cisplatin and its derivatives.

The AminoMedix™ compositions may be used for kidney protection during imaging using radiocontrast agents and gadolinium-based contrast agents. The AminoMedix™ compositions may be used for kidney protection during imaging using multimodality compounds such as but not limited to radiolabeled/optical probes.

The components in the AminoMedix™ compositions may be used at any suitable amounts or concentrations. One skilled in the art would appreciate that a suitable amount may depend on the patients and the therapeutic or diagnostic agents being used. Optimizing a suitable amount involves routine techniques, and one skilled in the art would be able to find a suitable amount without undue experimentation. For example, the Amifostine in the AminoMedix™ composition may be used at a concentration of from about 10 mg to about 500 mg per kilogram of body weight, preferably from about 18 mg to about of 180 mg per kilogram of body weight. The at least one amino acid in the AminoMedix™ compositions may be used in a range from about 50 mg to about 1 g per kilogram of body weight, preferably from about 150 mg to about 700 mg per kilogram of body weight. The AminoMedix™ compositions may be used with or without one or more active compounds at a concentration from about 18 mg to about 180 mg per kilogram of body weight. For example, the AminoMedix™ compositions may comprise Amifostine used in a concentration of 18 mg to about of 500 mg per kilogram of body and Arginine used in the range of amount of 150 mg to about 800 mg per kilogram body and Lysine used in the amount of 150 mg to about 800 mg, with or without one active compound used in concentration of 18 mg to about of 180 mg per kilogram of body weight.

The AminoMedix™ compositions may be used as protective agents prior to the administration of such therapeutic or diagnostic compounds, or they may be used as protective agents by co-administration with one or more other therapeutic or diagnostic compounds.

Embodiments of the invention will be illustrated with the following examples. These examples are for illustration only and they are not intended to limit the scope of the invention.

### EXAMPLES

The following examples are provided to illustrate the efficacies of AminoMedix™ compositions comprising Amifostine and at least one amino acid or oligomer, with or without one or more therapeutic or diagnostic compounds. These compositions may be used as kidney protective agents.

### Example 1

Kidney protection effects of Lysine-Arginine (12.4 mg/ml and 12.5 mg/ml, respectively) and commercially available Clinisol® and Gelofusine co-injected with ⁶⁸Ga-DOTATATE (40 uCi) are determined based on biodistribution studies in mice. Lysine-Arginine, Clinisol® and Gelofusine solutions were used as positive controls to determine the efficacies of compositions in accordance with embodiments of the invention.

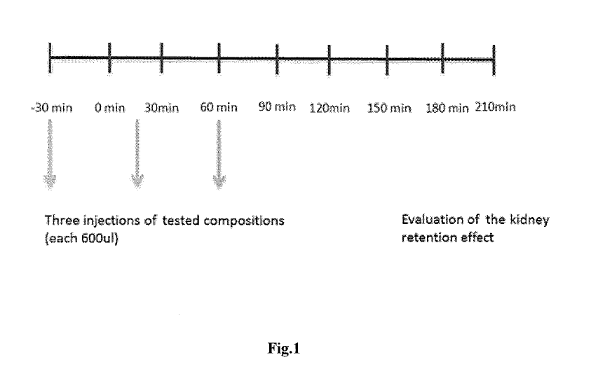

Mice (n=5/group, male 6-8 weeks old) were given tail intravenous injection (200 ul) of respective solutions, 30 min prior to the administration of ⁶⁸Ga-DOTATATE, followed by further injections at 15 min and 1 hr post injection. An example of application schedule is illustrated in FIG. 1.

Mice were then sacrificed by CO₂ asphyxiation, and kidneys were collected to determine the amounts of radiation absorbed, which are then compared to the total injected radioactive doses.

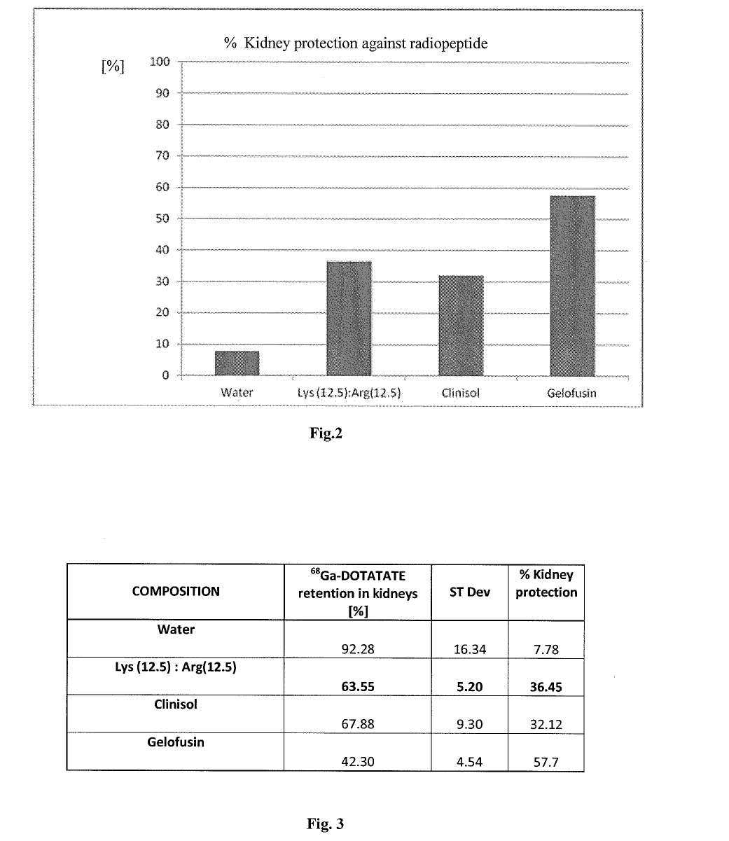

As shown in FIGS. 2 and 3, administration of a Lysine-Arginine composition (12.5 mg/ml and 12.5 mg/ml, respectively) provides radioprotection of kidneys by 36.45%. Clinisol® and Gelofusine injections protect kidneys by 32.12% and 57.7%, respectively.

### Example 2

Reductions of kidney uptakes of ⁶⁸Ga-DOTATATE by the AminoMedix™ compositions are tested. The AminoMedix™ compositions comprise Amifostine and at least one Amino acid. These compositions may be used with or without one or more therapeutic or diagnostic compounds.

The following AminoMedix™ compositions were tested:
Lysine: Arginine: Amifostine (7.5 mg/ml: 17 mg/ml: 0.63 mg/ml);
Lysine: Arginine: Amifostine (7.5 mg/ml: 12.5 mg/ml: 0.63 mg/ml);
Lysine: Arginine: Amifostine (12.5 mg/ml: 12.5 mg/ml: 0.63 mg/ml);
Lysine: Arginine: Amifostine (25 mg/ml: 25 mg/ml: 0.63 mg/ml);

AminoMedix™ compositions comprising Amifostine and at least one amino acid, with or without one or more therapeutic or diagnostic compounds, may be prepared in pharmaceutically accepted diluents, such as, but not limited to, water, saline and Ringer solution, and PBS, and the final pH values of the solutions may be adjusted to a value in the range of about 5.6-7.8.

Mice (n=5/group, male 6-8 weeks old) were given tail intravenous injection (200 ul) of respective solutions, 30 min prior to the administration ⁶⁸Ga-DOTATATE, followed by further injections 15 min and 1 hr post injection, as described in Example 1.

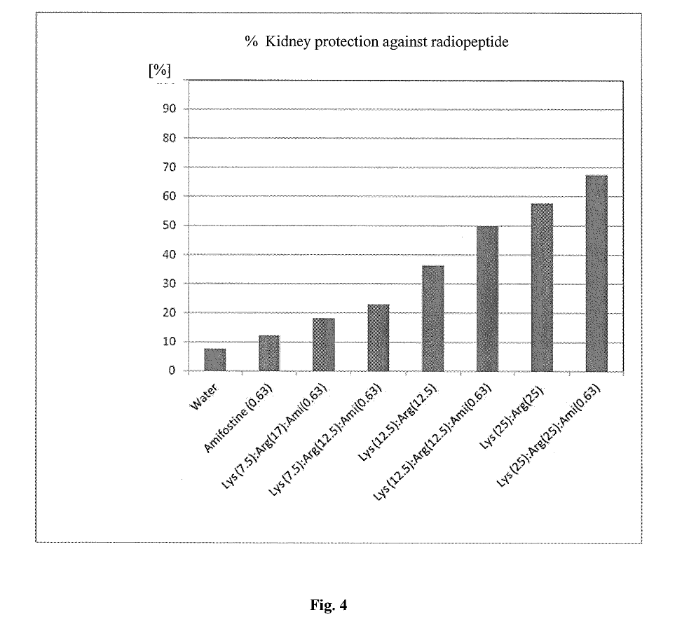

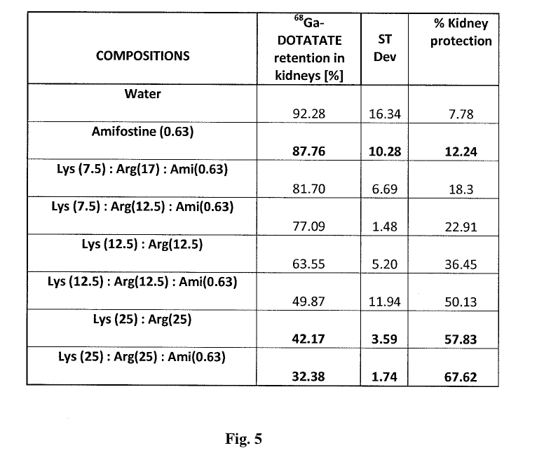

As shown in FIGS. 4 and 5, co-injection of AminoMedix™ composition comprising from Lysine: Arginine: Amifostine (25 mg/ml: 25 mg/ml: 0.63 mg/ml) had the highest radioprotective effect on the kidneys, up to 67.62%, while Lysine: Arginine (25 mg/ml: 25 mg/ml) used alone had a protective effect of 57.83%. There is a significant benefit in the addition of Amifostine, to the compositions of Lysine and Arginine, at a concentration of 0.63 mg/ml. That is, Amifostine further enhances the protective effects of lysine and arginine.

AminoMedix™ composition of Lysine:Arginine:Amifostine (12.5 mg/ml: 12.5 mg/ml: 0.63 mg/ml) is more efficient in kidney protection against radiopeptides than others. The kidney protective effects using this composition is 50.13%, while the effect using Lysine: Arginine (12.5 mg/ml:12.5 mg/ml) alone (i.e., without Amifostine) is only 36.45%.

Therefore, addition of Amifostine to the composition of Arginine and Lysine has a synergistic effect in the reduction of renal uptakes of radiopeptides, thereby improving the radioprotective properties of individual components.

### Example 3

This example tests the kidney protection effects by Amifostine and at least one amino acid, with or without one or more therapeutic or diagnostic compounds. The compositions were co-administrated with ⁶⁸Ga-DOTATATE

The following compositions of Amifostine and at least one Amino acid with or without one active compound were tested:
Amifostine (15 mg/ml);
Amifostine (0.63 mg/ml): Lysine (25 mg/ml): L-Histidine (25 mg/ml);
Probenecid (10 mg/ml); and
Amifostine (25 mg/ml): Lysine (25 mg/ml): Probenecid (10 mg/ml).

Mice (n=5/group, male 6-8 weeks old) were given tail intravenous injection (200 ul) of respective solutions, 30 min prior to the administration ⁶⁸Ga-DOTATATE, followed by further injections 15 min and 1 hr post injection, as described in Example 1.

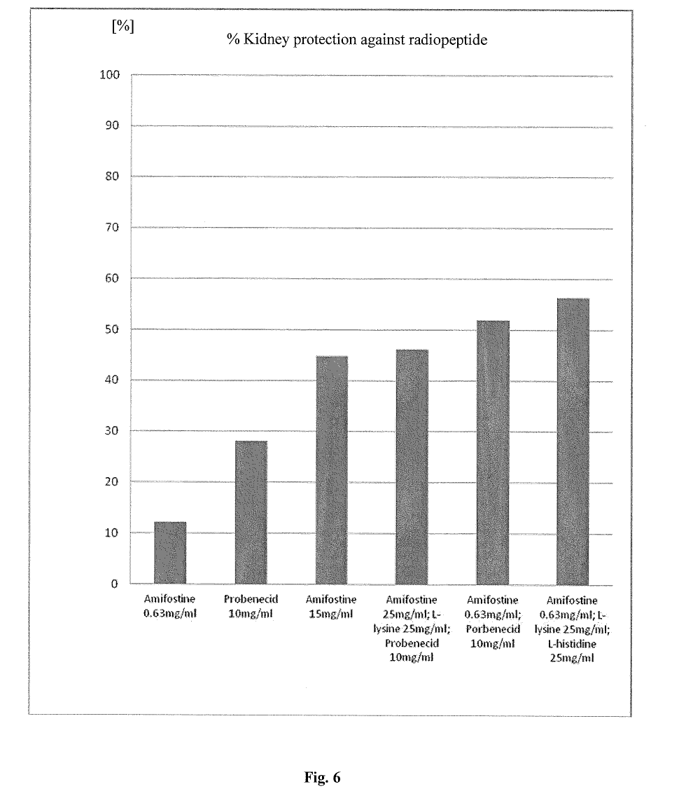

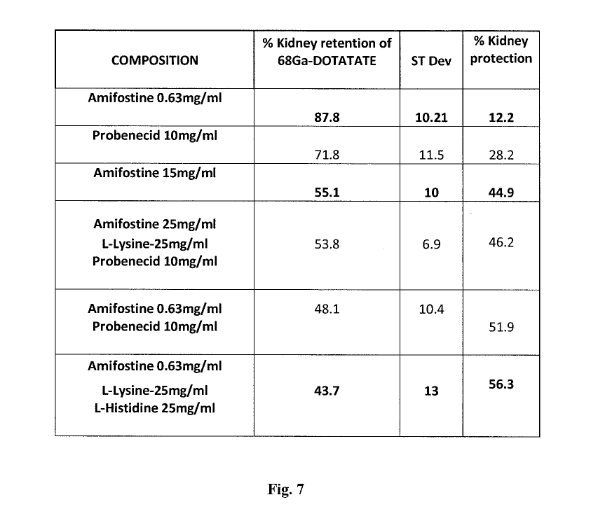

As shown in FIGS. 6 and 7, Amifostine used alone as kidney radiprotective agent provides protection of kidneys by only 12.2%, at a dose of 0.63 mg/ml. The effects of Amifostine in radioprotection of kidney are more pronounced in the presence at least one amino acid (Lysine, Histidine, Arginine), and in the presence of a pharmaceutically active compound (Probenecid). For example, co-administration of an AminoMedix™ composition consisting of Amifostine (0.63 mg/ml): Lysine (25 mg/ml) and L-Histidine (25 mg/ml) protects kidneys by 56.3%.

### Example 4

This example tests the kidney protection effects by Amifostine with at least one amino acid and Vitamin B12. The compositions were co-administrated with ⁶⁸Ga-DOTATATE

The following compositions of Amifostine and at least one Amino acid with or without one active compound (Vitamin B12) were tested:
Amifostine (0.63 mg/ml),
Amifostine (0.63 mg/ml): Lysine (25 mg/ml): L-Arginine (25 mg/ml): Vitamin B12 (1 mg/ml)
Amifostine (0.63 mg/ml): Lysine (12.5 mg/ml): L-Arginine (12.5 mg/ml): Vitamin B12 (1 mg/ml)

Mice (n=5/group, male 6-8 weeks old) were given tail intravenous injection (200 ul) of respective solutions, 30 min prior to the administration ⁶⁸Ga-DOTATATE, followed by further injections 15 min and 1 hr post injection, as described in Example 1.

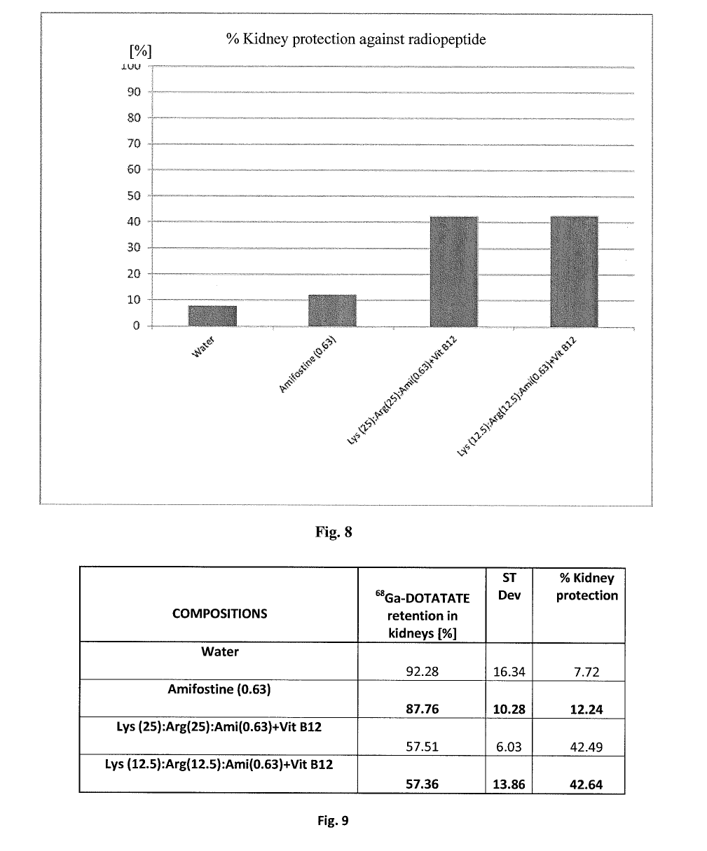

As shown in FIGS. 8 and 9, Amifostine used alone as kidney radioprotective agent provides protection of kidneys by only 12.24%, at a dose of 0.63 mg/ml. The effects of Amifostine in radioprotection of kidney are more pronounced in the presence at least one amino acid (Lysine, Arginine), and in the presence of a pharmaceutically active compound (Vitamin B12). For example, co-administration of an AminoMedix™ composition consisting of Amifostine (0.63 mg/ml): L-Lysine (25 mg/ml) and L-Arginine (25 mg/ml): Vit.B12 (1 mg/ml) protects kidneys by 42.49%.

### Example 5

This example tests the kidney protection effects by Clinisol® or Amifostine with at least one amino acid. The compositions were co-administrated with ⁶⁸Ga-DOTATATE

The following compositions of Clinisol® or Amifostine and at least one Amino acid were tested:
Amifostine (0.6 mg/ml)
Lys (0.21 mg/ml): Arg (2.5 mg/ml): Amif (0.63 mg/ml)
Lys (0.21 mg/ml): Arg (2.5 mg/ml): Amif (1 mg/ml)
Clinisol®
Lys (12.5 mg/ml):Arg(12.5 mg/ml)
Lys (2.5 mg/ml):Arg(2.5 mg/ml):Ami(0.63)
Lys (1.05 mg/ml): Arg (12.5 mg/ml): Amif (0.63 mg/ml)
Lys (2.1 mg/ml):Arg(25 mg/ml):Ami(0.63 mg/ml)

Mice (n=5/group, male 6-8 weeks old) were given tail intravenous injection (200 ul) of respective solutions, 30 min prior to the administration ⁶⁸Ga-DOTATATE, followed by further injections 15 min and 1 hr post injection, as described in Example 1.

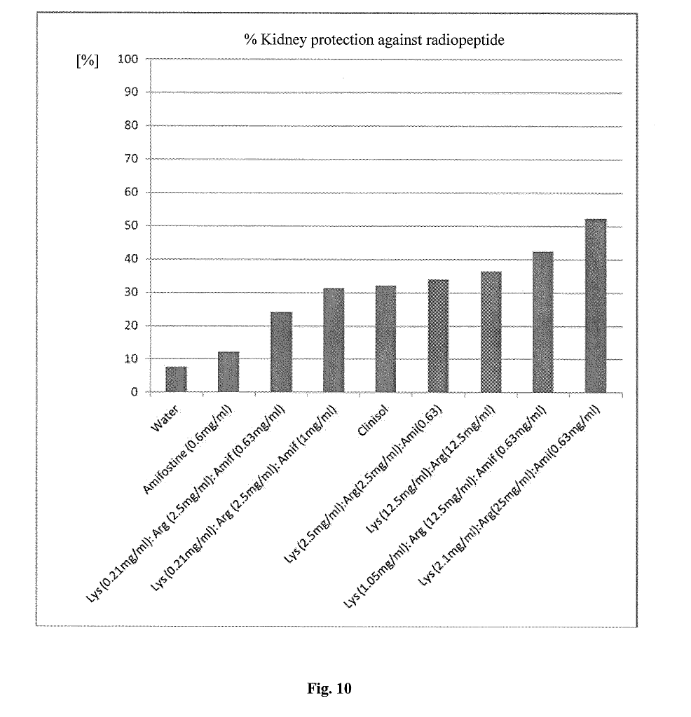

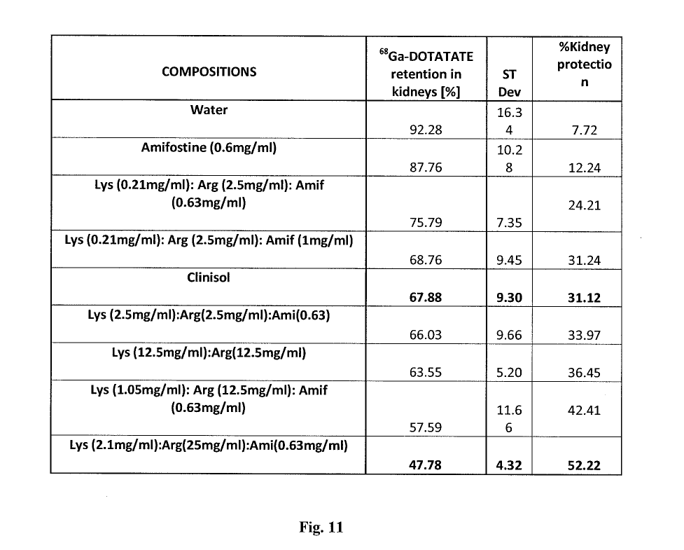

As shown in FIGS. 10 and 11, Clinisol provides protection of kidneys by only 31.12%. Co-administration of an AminoMedix™ composition consisting of Amifostine (0.63 mg/ml): L-Lysine (2.1 mg/ml) and L-Arginine (25 mg/ml) protects kidneys by 52.22%.

While the invention has been described with reference to a limited number of examples, one skilled in the art would appreciate that other modifications or variations are possible without departing from the scope of the invention. Therefore, the scope of protection should be limited only by the attached claims and the equivalents thereof.

### REFERENCES

Andreassen C N, Grau C, Lindegaard J C., Semin Radiat Oncol. 2003 January; 13(1):62-72.; Chemical radioprotection: a critical review of amifostine as a cytoprotector in radiotherapy.
Copp R R, Peebles D D, Soref C M, Fahl W E.; Int J Radiat Biol. 2013 February 20.; “Radioprotective efficacy and toxicity of a new family of aminothiol analogs.”
Culy C R, Spencer C M. “Amifostine: an update on its clinical status as a cytoprotectant in patients with cancer receiving chemotherapy or radiotherapy and its potential therapeutic application in myelodysplastic syndrome.” Drugs. 2001; 61(5):641-84.
Combination of amino acid solution and a gelatin derivative for inhibiting renal uptake EP 2021012 A1; Patent: WO2007137871A1; Inventors: Marion De Jong; P. Eric Krenning; Edgar Rolleman
Barone R, Pauwels S, De Camps J, Krenning E P, Kvols L K, Smith M C, Bouterfa H, Devuyst O, Jamar F.; Nephrol Dial Transplant. 2004 September; 19(9):2275-81. Epub 2004 Jul. 13.; “Metabolic effects of amino acid solutions infused for renal protection during therapy with radiolabelled somatostatin analogues.”
Bushnell D, Menda Y, O'Dorisio T, Madsen M, Miller S, Carlisle T, Squires S, Kahn D, Walkner W, Connolly M, O'Dorisio S, Karwal M, Ponto J, Bouterfa H.; Cancer Biother Radiopharm. 2004 February; 19(1):35-41; “Effects of intravenous amino acid administration with Y-90 DOTA-Phel-Tyr3-Octreotide (SMT487 [OctreoTher) treatment.”
Duncan J R, Stephenson M T, Wu H P, Anderson C J.; Cancer Res. 1997 Feb. 15; 57(4):659-71.; Indium-111-diethylenetriaminepentaacetic acid-octreotide is delivered in vivo to pancreatic, tumor cell, renal, and hepatocyte lysosomes.
Hammond P J, Wade A F, Gwilliam M E, Peters A M, Myers M J, Gilbey S G, Bloom S R, Calam J.; Br J Cancer. 1993 June; 67(6):1437-9 “Amino acid infusion blocks renal tubular uptake of an indium-labelled somatostatin analogue.
Melis M, Krenning E P, Bernard B F, Barone R, Visser T J, de Jong M.; Eur J Nucl Med Mol Imaging. 2005 October; 32(10):1136-43. Epub 2005 May 24.” Localisation and mechanism of renal retention of radiolabelled somatostatin analogues.”
Melis M, Valkema R, Krenning E P, de Jong M.,; J Nucl Med. 2012 May; 53(5):749-53; “Reduction of renal uptake of radiolabeled octreotate by amifostine coadministration”.
Medicament for the administration of amifostine and related compounds, Patent application, WO2002062350A1
Moestrup S K, Birn H, Fischer P B, Petersen C M, Verroust P J, Sim R B, Christensen E I, Nexø E.; Proc Natl Acad Sci USA. 1996 Aug. 6; 93(16):8612-7.; “Megalin-mediated endocytosis of transcobalamin-vitamin-B12 complexes suggests a role of the receptor in vitamin-B12 homeostasis.”
Nagai J, Takano M.; Expert Opin Drug Metab Toxicol. 2010 September; 6(9):1125-38. “Molecular-targeted approaches to reduce renal accumulation of nephrotoxic drugs.”
Pool S E, Krenning E P, Koning G A, van Eijck C H, Teunissen J J, Kam B, Valkema R, Kwekkeboom D J, de Jong M.; Semin Nucl Med. 2010 May; 40(3):209-18.; “Preclinical and clinical studies of peptide receptor radionuclide therapy.”
Rolleman E J, Valkema R, de Jong M, Kooij P P, Krenning E P.; Eur J Nucl Med Mol Imaging. 2003 January; 30(1):9-15. Epub 2002 Oct. 29.; Safe and effective inhibition of renal uptake of radiolabelled octreotide by a combination of lysine and arginine.
Santini V.; Expert Opin Pharmacother. 2001 “Amifostine: chemotherapeutic and radio-therapeutic protective effects.;
Vegt E, Eek A, Oyen W. J. G, Jong deM., Gotthardt M., Boerman O.; Eur J Nucl Med Mol Imaging. 2010 February; 37(2): 226-234.; “Albumin-derived peptides efficiently reduce renal uptake of radiolabelled peptides”

What is claimed is:

1. A composition for kidney protection during imaging or therapy using a radiolabeled and/or a non-radiolabeled compound, comprising:
Amifostine; and
at least one positively charged amino acid or an oligomer comprising positively charged amino acids.

2. The composition of claim 1, wherein the at least one amino acid or the oligomer is selected from D-Arginine, L-Arginine, or poly-Arginine.

3. The composition of claim 1, wherein the at least one amino acid or the oligomer is selected from D-Lysine, L-Lysine, poly-Lysine, or Ornithine.

4. The composition of claim 1, further comprising a pharmaceutically acceptable excipient, diluent, carrier, or a combination thereof.

5. The composition of claim 1, further comprising a pharmaceutically active compound selected from Vitamin B12, Carnitine, L-Histidine, D-Histidine, Probenecid, Albumin or a proteolysis product thereof, Globulin or a proteolysis product thereof, a peptide fragment of cytochrome c, or a peptide fragment of an actin-regulating protein.

6. A method for kidney protection, comprising administering a composition of claim 1 to a subject during imaging and/or therapy using a radioisotope labeled agent.

7. The method of claim 6, wherein the radioisotope labeled agent comprises a radionuclide selected from 177Lu, 111In, 90Y, 117mSn, 45Ti, 59Fe, 60Cu, 61Cu, 62Cu, 64Cu, 67Ga, 68Ga, 89Sr, 99mTc, 149Pm, 153Gd, 153Sm, 186Re, 188Re, 211 At, 212Bi, 225 Ac, 125I, 123I, 32P, or 223Ra.

8. The method of claim 6, wherein the radioisotope labeled agent is a peptide, a protein, an antibody, a carbohydrate, a glycopeptide, a urea-derivative, a nucleotide, a nucleoside, a heterocyclic compound, a plant extract, a nanoparticle, a polymer, a nanomaterial, or a composition comprising a plant-derived compound.

9. The method of claim 6, wherein the therapy is peptide receptor targeted therapy (PRRT) using a radiolabeled agonist or antagonist.

10. The method of claim 9, wherein the agonist or antagonist targets a somatostatin-receptor.

11. The method of claim 10, wherein the agonist or antagonist is a DOTA-, DTPA-, or NOTA-based derivative.

12. The method of claim 11, wherein the DOTA-, DTPA-, or NOTA-based derivative is selected from DOT AT ATE, DOTATOC, DOTANOC, DOTA-BASS, JR-11 or DOTA-BIM.

13. The method of claim 6, wherein the therapy uses an antibiotic or a non-radiolabeled nephrotoxic drug.

14. The method of claim 6, wherein the therapy is chemotherapy.

15. The method of claim 6, wherein the imaging uses a radiocontrast agent or a gadolinium-based contrast agent.

16. The method of claim 6, wherein the imaging uses a multimodality compound having a radiolabeled probe and an optical probe.

17. The method of claim 6, wherein the composition comprises Amifostine in an amount of about 2 mg to about of 180 mg per kilogram of body weight and the at least one amino acid or the oligomer in an amount of about 150 mg to about 1,000 mg per kilogram of body weight.

18. The method of claim 6, wherein the composition further comprises an active compound selected from Vitamin B12, Carnitine, L-Histidine, D-Histidine, Probenecid, Albumin or a proteolysis product thereof, Globulin or a proteolysis product thereof, a peptide fragment of cytochrome c, or a peptide fragment of an actin-regulating protein.
